(19) United States
(12) Patent Application Publication
(10) Pub. No.: US 2007/0248723 A1
(43) Pub. Date: Oct. 25, 2007

(54) Device for Killing Living Microorganism

(76) Inventor: Bing-Yang Yao, Sanchong (TW)

Correspondence Address: Heng-Yi Lin, 7F.-6, No.17, Sec. 1, Chengde Rd., Taipei, 10351, omitted

(21) Appl. No.: 11/739,098

(22) Filed: Apr. 24, 2007

(30) Foreign Application Priority Data
Apr. 25, 2006 (TW) 95114603

(51) Int. Cl. A23L 3/32

(52) U.S. Cl. 426/237

(57) ABSTRACT

A device for killing living microorganisms in an amount of raw meat is described in order to make the raw meat capable of being eaten without causing infection. The non-heating for killing living microorganisms comprises an enclosure, a carrier, a light source and a rotator. The carrier, in the enclosure, is for carrying the raw meat. The light source, in the enclosure, is for emitting light killing the living microorganisms in the raw meat. The rotator rotates the raw meat so that the rotated raw meat can be fully exposed to the emitted light.

### FIELD OF THE INVENTION

This invention relates generally to a device for killing living microorganisms, and more particular, to a device for killing living microorganisms of an amount of raw food.

### BACKGROUND

In recent years, raw foodism is being raised in Europe and US. In this revolution, people imitate barbarians to eat raw food and raw vegetables without cooking them.

In the past, people heated food before eating because they wanted to kill microorganisms in the food. In a raw food diet, many microorganisms may cause infection or diseases. Heating food may kill the microorganisms. However, the heat also destroys many substances such as natural enzyme, amino acid, vitamin C and the like beneficial to people. Some mineral substance in an “organic status” is easily absorbed by human, but often heated and therefore destroyed to reduce its nutritional value. The heated mineral substance is alternatively in an “inorganic status”.

American ministry of agriculture has ever published a specialized book. The book describes in detail that the numbers of nutrients in a raw food before and after being cooked are quite different. Accordingly, eating raw food prevents the food from losing nutrients, and relatively increases the nutrients absorbed by human, and relatively reduces the consumption of foods.

Primitive Eskimo eat raw meat more than cooked meat. The reason may be that cooking wastes the fuel quite a lot and is not easy in a frozen and snow-covered land. Eskimo kill and then immediately eat a seal, walrus or fish once they are hunt. Such diet custom makes the Eskimo healthy and strong without common problems such as coronary arteriosclerosis and similar cardiovascular diseases. The rates for Eskimo to get modern diseases are often lower than people in advanced countries. After the western civilization spreads to the Eskimo's life, the Eskimo changes their custom of eating raw food. At the same time, the rates for the Eskimo to get modern diseases are obviously raised.

Many foreign experiments were performed to animals to see the difference between eating raw food and cooked food. Those experiments made conclusions substantially the same as the conclusion of the Eskimo's change of diet custom. That is why the raw-food revolution is being raised recently. There are many advantages for people to eat raw food. Why most of them are hesitant to do it? The major reason may be the microorganisms of the raw foods cannot be effectively removed. For example, in September 2006, a poison spinach incident occurred in US. The tragedy killed several people due to they ate spinach which was polluted with Escherichia coli. Keeping nutrients of raw foods and making them safe and healthy for people to eat are therefore topics that the present invention intends to improve.

A conventional food vessel delaying food acidification was described in Taiwan Patent No. 588736. In this food vessel, some microorganisms may be killed to delay the food acidification, but most of the microorganisms cannot be effectively killed. Accordingly, this delay cannot make sure that people won't be infected or become ill if they eat a raw food which is kept in such a food vessel. The raw food, such as a sashimi, is kept in the same position without motion in a microorganism killing process. In this case, some microorganisms on a surface of the raw food may be killed, but other microorganisms on the other surfaces of the raw food may not be killed or may even grow to spread out the whole surfaces.

### SUMMARY

One purpose of the present invention may be to provide a first device for sufficiently killing living microorganisms. The first device has a light source generating light capable of killing the living microorganisms. An object in the first device is rotated to fully expose to the light, so that the living microorganisms may be fully killed.

Another purpose of the present invention may be to provide a second device for sufficiently killing living microorganisms. The second device comprises an indicator indicating the operating status/working function. By the indicator, a user may simply know the working function very well. This indicator avoids being unaware of the second device cannot kill living microorganisms any more.

The other purpose of the present invention may be to provide a third device for sufficiently killing living microorganisms. The third device may comprise a reflector disposed on an inner sidewall of an enclosure. The reflector reflects light to sufficiently emit on an object having the living microorganisms, for sufficiently killing the living microorganisms by the light.

According to the purposes of the present invention, a device for killing living microorganisms may comprise a motor, an enclosure, a rotator and a light source for killing the microorganism. The enclosure has a containing space. The rotator is disposed in the enclosure and has a fixing mechanism retaining an object. The light source is also disposed in the enclosure. The light source generates light being fully emitted to the rotator in the containing space. When a user initiate the device for killing the living microorganisms of the present invention, the fixed object can be driven and rotated by a fixing mechanism of the rotator. This is achieved by a power provided by the motor. The light is emitted to the object in the rotator to sufficiently kill the living microorganisms of the object such as a raw food. The raw food is safe to be eaten without being cooked. If the object is an object directly contacting with a human body such as a tooth brush or a pacifier, the living microorganisms on the object can also be fully killed.

### BRIEF DESCRIPTION OF THE DRAWINGS

FIG. 1 schematically shows a device for sufficiently killing living microorganisms according to the present invention;

FIG. 2A to FIG. 2B schematically show the implementation of a device for sufficiently killing living microorganisms according to the present invention; and

FIG. 3A to FIG. 3B schematically show a rotator according to the present invention.

### DETAILED DESCRIPTION OF THE INVENTION

A device for sufficiently killing living microorganisms is disclosed. In the following detailed description, numerous specific details are set forth to provide a full understanding of the present invention. It will be obvious, however, to one ordinarily skilled in the art that the present invention may be practiced without some of these specific details. In other instances, well-known structures and techniques have not been shown in detail so as to avoid unnecessarily obscuring the present invention.

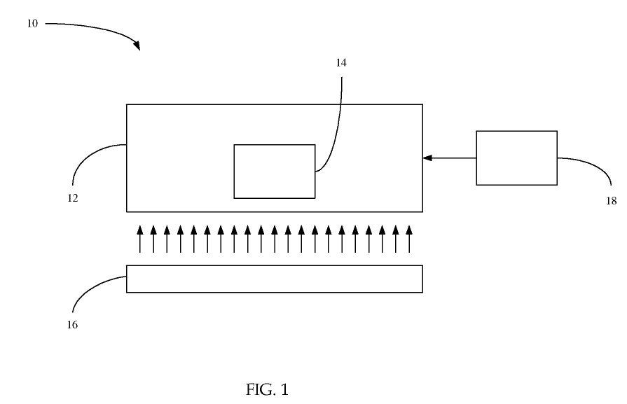

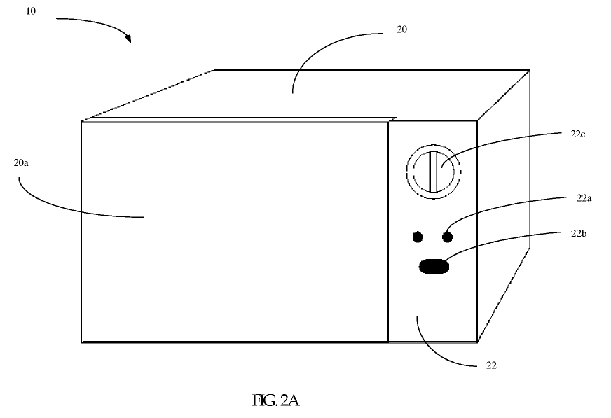

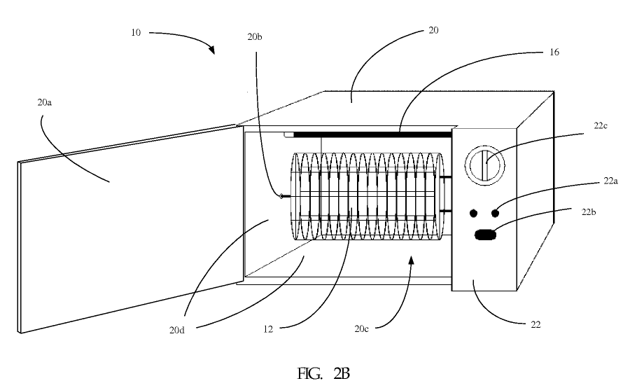

Please refer to FIG. 1, it schematically shows a device for sufficiently killing living microorganisms in an object according to the present invention. As shown in FIG. 1, the device 10 for sufficiently killing the living microorganisms may comprise a motor 18, an enclosure 20 (as shown in FIG. 2A or FIG. 2B), rotator 12, a light source 16 for killing the living microorganisms. The light source 16 for killing the living microorganisms may be a UV light or a nanometer photocatalyst. The motor 18 generates a power and transmits the power to the rotator 12 by a transmitting mechanism (such as a gear set not shown). The rotator 12 is therefore driven to self-rotate and to rotate the object 14.

Simply, by using the motor 18 and a transmitting mechanism to rotate the object 14, light source 16 of the device for sufficiently killing the living microorganisms emits light onto the object 16 enough to sufficiently kill the living microorganisms in the object 14, because the rotated object 14 can be fully exposed to the emitted light. As a result, the object 14, such as a raw food, can be safely eaten without being cooked. The object 14 may alternatively be a raw food, knife, toothbrush, cleaning rag, milk bottle, pacifier or any object which may be fixed by a fixing mechanism (lately described) of the rotator 12.

To clearly understand how the object 14 is rotated, an example combining a microwave oven and a chicken-rotated roaster is described hereinafter. Please refer FIG. 2A to FIG. 2B, they schematically show the implementation of a device for sufficiently killing living microorganisms according to the present invention. As shown in FIG. 2A, the enclosure 20 of the device 10 for killing the living microorganisms looks like a microwave having a door and a control panel. The enclosure 10 may include a door 20a and a control panel 22. The control panel may have an opening button 22b, a rotary switch 22c and an indicator 22a indicating the operating status/working ability.

The size of the enclosure 20 is designed according to the user of the device 10. The user may be a member of a family or a company. The quantity of the indicators may be corresponded to the quantity of the light source 16 for killing the living microorganisms. Therefore, the user may easily understand the operating status/working ability of the light source 16 for killing the living microorganisms according to that the indicator 22a is light or dark and its brightness or color (for example, red indicates the device gets something wrong, green indicates the device is normal) or twinkling status. Such an indicator 22a prevents the user from being unaware of a breakdown of the light source 16.

The rotary switch 22c may be for setting a working time of the device 10. The opening button 22b may be pressed to open the door 20a as shown in FIG. 2B. The light source 16 for killing the living microorganisms is temporarily stopped working to prevent human body from being hurt by any emitted light. The light source 16 is kept working after the door 20a is closed. When the door 20a is opened, it is observed that the enclosure 20 comprises a containing space 20a, a reflector 20d on an inner sidewall of the enclosure, assembly of retainer bases 20b, and the rotator 12 and light source 16 both disposed in the containing space 20c.

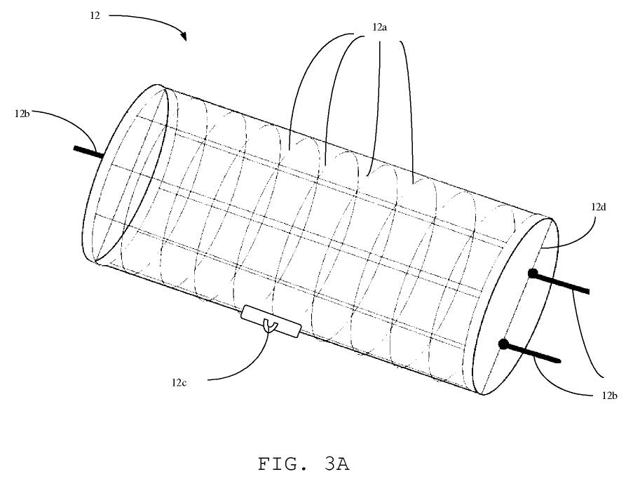

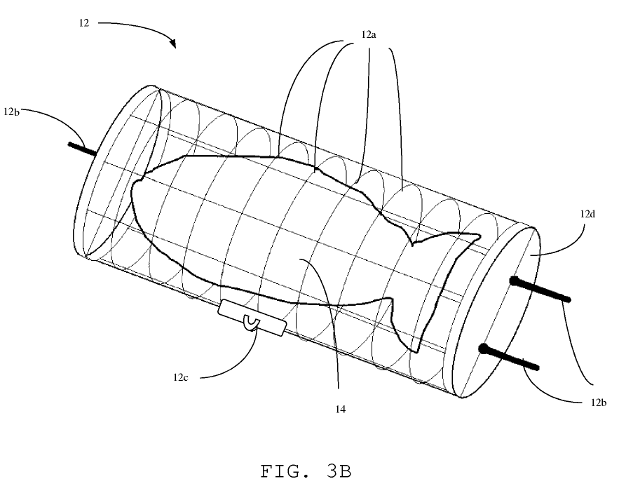

Although the quantity and the position of the light source 16 for killing the living microorganisms are not limited, the light generate by the light source 16 should be enough to fully emit to the rotator 12 and especially onto the object 14 in the rotator 12 (as shown in FIG. 3B). By the emitted light, the living microorganisms may be fully killed. When the rotator 12 is driven by the motor 18 (i.e., the object is rotated by the rotator 12).

In addition to the rotator 12 being emitted for killing the living microorganisms, a reflector 20d is disposed on the inner sidewall of the enclosure 20. The reflector reflects the light from the light source 16 to the rotator 12 and the object 14 in the containing space 20c for more emission. The reflector 20d may be a mirror or a metal surface having reflecting function. The reflector 20d of the reflector may be processed with a nanometer photocatalyst by, for example, being coated with a titanium dioxide layer. The titanium dioxide layer is activated to generate activators for killing the living microorganisms and to have hydrophilicity, when the reflector 20d is emitted with the light from the light source 16 and when the reflector 20d reflects the emitted light. The door 20a may be opened for the user to take or to put the rotator 12 after the object 14 is disposed in the rotator 12.

The following will describe how to take or to put the rotator 12 into the enclosure 20. Please refer to FIG. 3A to FIG. 3B, they schematically show a rotator according to the present invention. As shown in FIG. 3A, the rotator 12 may be a drum made of a wire netting 12a. Through the wire netting 12a, the light from the light source 16 can be emitted onto the object 14. On the other hand, the object 14 is fixed by the wire netting 12a, rather than not fixed by the rotator 12 without a wire netting. The wire netting 12a may serves as a fixing mechanism for retaining and fixing the object 14.

However, the rotator may be made by other fixing mechanism than a wire netting. Alternatively, other fixing mechanism, which fixes the object 14 so that the light may emit onto the object 14 in the rotator 12, is suitable in the present invention. If the object 14 needs to be put into the enclosure, open the rotator 12 which can be half opened along a thin slit 12d by a retaining ring 12c of the rotator 12 (i.e., the wire netting 12a has a structure which can be half opened). The rotator 12 is closed after the object 14 is put onto/taken out of the enclosure 20 again by the retaining ring 12c. When the rotator 12 containing the object 14 needs to be put into the enclosure 20, it is shown as FIG. 2B.

The enclosure 20 has assembly of retainer bases 20b opposite to the containing space 20c. One of the retainer bases is disposed on one side. Other retainer bases on the other side are not shown, but are disposed opposite to the rotator 12 and essentially have a quantity of two. The rotator 12 has two sides and two retainers 12b respectively set on the two sides. On one side, a single retainer is set. The rotator 12 is fixed in the enclosure 20 by locating the retainers 12b into the assembly of retainer bases 20b. The rotator 12 is self-rotated by a power through a driving mechanism and from the motor 18. The contained object 14 is also simultaneously rotated by the rotator.

The device for killing the living microorganisms of the present invention may comprise only the enclosure 20, a carrier (which may substantially equivalent to a rotator 12 being swung and not driven by the motor 18), and a light source 16. In this case, the light source 16 must generate the light fully emitting on the object 14 on the carrier, or alternatively, must be reflected to other portion of the swung object 14 by the above-mentioned reflector 20d. The other portion of the swung object 14 is not directly exposed to the emitted light, but is still exposed to the reflected light so that the living microorganisms may be fully killed.

In addition to the reflector 20d, the device may further comprise another light source disposed opposite to the light source 16. It prevents one side of the object 14 on the carrier from being not exposed to the emitted light from the light source 16.

To achieve the above results, a rotary switch (not shown) may be added to the control panel 22. After the rotary switch is pressed down, the motor 18 may not drive the rotator 12. As for the mentioned carrier, it is suspended, and is not driven by the motor 18.

Obviously, many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A non-heating device for killing living microorganisms in an amount of raw meat in order to make the raw meat capable of being eaten without causing infection, the non-heating device for killing living microorganisms comprising:
an enclosure;
a carrier, in the enclosure, for carrying the raw meat;
a light source, in the enclosure, for emitting light killing the living microorganisms in the raw meat; and
a rotator rotating the raw meat so that the rotated raw meat can be fully exposed to the emitted light.

2. A device for killing living microorganisms in an object, comprising:
a motor;
an enclosure;
a rotator fixing the object in the enclosure; and
a light source, in the enclosure, for emitting light capable of killing the living microorganisms in the object, wherein the rotator is driven by the motor to rotate the object, so that the rotated object is exposed to the emitted light enough to kill the living microorganisms therein.

3. The device of claim 2, wherein the object is a raw food, knife, toothbrush, cleaning rag, milk bottle or pacifier.

4. The device of claim 2, wherein the light source is a UV light or a nanometer photocatalyst.

5. The device of claim 2, wherein the light source in the enclosure comprises a UV light and a nanometer photocatalyst in the enclosure.

6. The device of claim 2, further comprising a control panel disposed out of the enclosure, wherein the control panel comprises an indicator.

7. The device of claim 2, wherein the enclosure comprises a door, and wherein the door is opened for a user to take the rotator.

8. The device of claim 2, wherein the enclosure has an inner surface and a reflector on the inner surface, and wherein the reflector is for reflecting the emitted light to the object.

9. The device of claim 2, wherein the reflector is coated with a titanium dioxide layer, and wherein the titanium dioxide layer is activated to kill the living microorganisms and to have hydrophilicity when the reflector reflects the emitted light.

10. The device of claim 2, wherein the enclosure has a containing space and has assembly of retainer bases opposite to the containing space, and wherein the rotator has two sides and two retainers respectively set on the sides, and wherein the rotator is fixed in the enclosure by locating the retainers into the assembly of retainer bases.

11. The device of claim 2, wherein the rotator is a drum made of a wire netting, and wherein the light is emitted onto the object through the wire netting.

12. The device of claim 2, wherein the rotator is a drum comprising a wire netting and a retaining ring, and wherein the retaining ring is for a user to partially open the wire netting.

13. A device for killing living microorganisms in an object, comprising:
an enclosure having a containing space;
a carrier, in the enclosure, having a fixing device containing the object; and
a first light source, in the enclosure, for emitting light for emitting light capable of killing the living microorganisms in the object.

14. The device of claim 13, wherein the object is a raw food, knife, toothbrush, cleaning rag, milk bottle or pacifier.

15. The device of claim 13, wherein the first light source is a UV light or a nanometer photocatalyst.

16. The device of claim 13, wherein the first light source in the enclosure comprises a UV light and a nanometer photocatalyst in the enclosure.

17. The device of claim 13, further comprising a control panel disposed out of the enclosure, wherein the control panel comprises an indicator.

18. The device of claim 13, wherein the enclosure comprises a door, and wherein the door is opened for a user to take or put the rotator.

19. The device of claim 13, wherein the enclosure has an inner surface and a reflector on the inner surface, and wherein the reflector is for reflecting the emitted light to the object.

20. The device of claim 19, wherein the reflector is coated with a titanium dioxide layer, and wherein the titanium dioxide layer is activated to kill the living microorganisms and to have hydrophilicity when the reflector reflects the emitted light.
